(19) United States
(12) Patent Application Publication
(10) Pub. No.: US 2009/0088584 A1
(43) Pub. Date: Apr. 2, 2009

(54) PROCESS FOR PREPARING 3, 4-DISUBSTITUTED PHENYLACETIC ACIDS AND NOVEL INTERMEDIATES

(76) Inventors: John-Matthias Wiegand, Walding (AT); Carsten Schafer, Linz (AT); Melanie Palaoro, Leonding (AT); Wolfgang Skranc, Vienna (AT); Oliver Maurer, Kirchberg-Thening (AT)

Correspondence Address: NIXON & VANDERHYE, PC, 901 NORTH GLEBE ROAD, 11TH FLOOR, ARLINGTON, VA, 22203, US

(21) Appl. No.: 12/280,249

(22) PCT Filed: Jan. 22, 2007
(86) PCT No.: PCT/EP2007/000498

§ 371 Date: Nov. 18, 2008

(30) Foreign Application Priority Data
Feb. 22, 2006 (AT) A 287/2006

(51) Int. Cl. C07C 319/02; C07C 255/01; C07C 327/44

(52) U.S. Cl. 558/388; 562/426; 564/74

(57) ABSTRACT

A process for preparing 3,4-disubstituted phenylacetic acids of the formula (I) in which X is fluorine, chlorine, bromine or iodine and R is C₁-C₄-alkylthio, C₁-C₄-alkylsulfonyl or C₁-C₄-alkylsulfoxide, starting from a 2-halo-C₁-C₄-alkylthiobenzene of the formula (II) in which X is as defined above and R1 is C₁-C₄-alkylthio.

3,4-Disubstituted phenylacetic acids, for instance 3-halo-4-alkylthiophenylacetic acids, 3-halo-4-alkylsulfonyl phenylacetic acids or 3-halo-4-alkylsulfoxide phenylacetic acids, are valuable intermediates for the preparation of pharmaceuticals and active agrochemical ingredients.

The literature already discloses various preparation methods.

For instance, WO 00/58293 discloses a 4-stage process starting from 2-chloromethylthiobenzene and chlorooxoacetate, which are converted by means of Friedel-Crafts acylation. In the second step, reduction is then effected by means of sodium borohydride. The third step is then an acylation, which is followed by a reduction by means of samarium iodide to give the corresponding 3,4-disubstituted phenylacetic ester. Disadvantages in this process are the relatively large amounts of AlCl₃ in the first step and of samarium iodide in the last step, and the relatively low yields. A further disadvantage is the H₂ evolution in the course of reduction with NaBH₄.

WO 02/46173 likewise discloses a process for reacting 2-chloromethylthiobenzene and chlorooxoacetate. The first step is again the Friedel-Crafts acylation. This is followed by a hydrolysis and a Wolf-Kishner reduction by means of hydrazine hydrate.

In this process, the relatively large amounts of AlCl₃ in the first step and additionally the relatively large amounts of hydrazine hydrate used in the last step, and also the initially very low temperature of −50° C. in the last step, are likewise highly disadvantageous.

The toxicity and risk of decomposition of hydrazine hydrate is also highly disadvantageous.

It was an object of the present invention to provide a process for preparing 3,4-disubstituted phenylacetic acids which, starting from 2-haloalkylthiobenzene, avoids the previous disadvantages of the known processes and affords the desired phenylacetic acids in high yields and purities.

Unexpectedly, this object is achieved by a process via novel intermediate compounds.

The present invention therefore provides a process for preparing 3,4-disubstituted phenylacetic acids of the formula (I)

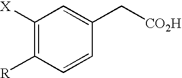

in which X is fluorine, chlorine, bromine or iodine and R is C₁-C₄-alkylthio, C₁-C₄-alkylsulfonyl or C₁-C₄-alkylsulfoxide, wherein a 2-halo-C₁-C₄-alkylthiobenzene of the formula (II)

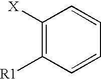

in which X is as defined above and R1 is C₁-C₄-alkylthio
a) is converted by means of the Blanc reaction with formaldehyde and HCl in the presence of a catalyst to the corresponding 3-halo-4-C₁-C₄-alkylthiobenzyl chloride of the formula (III)

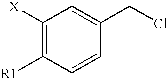

in which X and R1 are each as defined above, which is converted by a Kolbe nitrile synthesis with an alkali metal cyanide to the corresponding phenylacetonitrile of the formula (IV)

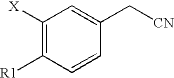

in which X and R1 are each as defined above, which is followed by hydrolysis to the phenylacetic acid of the formula (Ib)

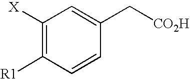

in which X and R1 are each as defined above, or
b) is converted by means of Friedel-Crafts acylation with acetyl chloride or acetic anhydride in the presence of aluminum chloride, iron (III) chloride, tin (IV) chloride or zinc chloride as a catalyst to the corresponding acetophenone of the formula (V)

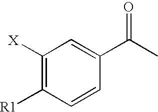

in which X and R1 are each as defined above, which is converted by a Willgerodt-Kindler reaction with sulfur and an amine of the formula HNR2R3 in which R2 and R3 are each independently a C₁-C₆-alkyl radical or together form a C₂-C₆-alkylene radical which may be interrupted by a heteroatom from the group of O, N or S to the corresponding thioamide of the formula (VI)

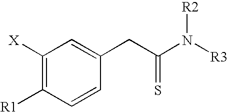

in which X, R1, R2 and R3 are each as defined above, which is then followed by hydrolysis to the phenylacetic acid of the formula (Ib)

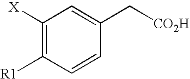

in which X and R1 are each as defined above, and, if appropriate, after a) or b), the R1 radical of the phenylacetic acid of the formula (Ib) is converted by oxidation to a C₁-C₄-alkylsulfonyl or C₁-C₄-alkylsulfoxide radical.

In the process according to the invention, 3,4-disubstituted phenylacetic acids of the formula (I) are prepared.

In the formula (I), X is a halogen radical from the group of chlorine, bromine, fluorine and iodine. X is preferably chlorine or bromine, more preferably chlorine.

The R radical may be C₁-C₄-alkylthio, C₁-C₄-alkylsulfonyl or C₁-C₄-alkylsulfoxide.

C₁-C₄-Alkyl is understood to mean a linear or branched alkyl radical which has from 1 to 4 carbon atoms and may optionally be substituted, for instance methyl, trifluoromethyl, ethyl, i-propyl, n-propyl, n-butyl, tert-butyl, etc.

The starting compound used for the process according to the invention is a 2-halo-C₁-C₄-alkylthiobenzene of the formula (II)

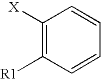

in which X is as defined above and R1 is C₁-C₄-alkyl-thio.

These compounds are commercially available or can be prepared according to the prior art (for example: WO 04/52869; WO 03/95438, WO 02/46173 or WO 00/58293).

In variant a), a Blanc reaction is first effected with formaldehyde and HCl in the presence of a catalyst to give the corresponding 3-halo-4-C₁-C₄-alkylthiobenzyl chloride of the formula (III).

Suitable catalysts are Lewis acids or mineral acids, for instance zinc chloride, aluminum chloride, PCl₃, POCl₃, sulfuric acid or phosphoric acid.

Formaldehyde may be used as an aqueous solution or as paraformaldehyde.

The amount of formaldehyde used is 1.5-5 equivalents based on the compound of the formula (II).

The catalyst is used in an amount of 0.1-1 equivalent based on the compound of the formula (II), preferably of 0.2-0.8 equivalent.

The catalyst used is preferably zinc chloride.

Hydrochloric acid may be used as a gas or an aqueous solution in an amount of 1.5-10 equivalents based on the compound of the formula (II).

The reaction temperature for this step is from 30 to 105° C., preferably from 40 to 60° C.

To isolate the 3-halo-4-C₁-C₄-alkylthiobenzyl chloride of the formula (III), on completion of reaction, the organic phase is removed and if appropriate washed with water, and unconverted starting materials are if appropriate removed by distillation.

The remaining distillation bottoms which comprise the desired compound may be used directly for the next step without further purification.

The purity of the benzyl chloride can be increased further if appropriate by distillation.

In the next step, either the distillation bottoms from the first step which comprise the benzyl chloride or further-purified benzyl chloride is used as the starting compound.

In the second step, a nitrile-Cl exchange is then effected, the corresponding phenylacetonitrile of the formula (IV) being obtained by reaction with an alkali metal cyanide.

Suitable alkali metal cyanides are preferably sodium cyanide or potassium cyanide.

The cyanide is used in an amount of 1-2 equivalents, preferably of from 1.01 to 1.5 equivalents, based on the benzyl chloride.

The reaction is effected, if appropriate, in the presence of a phase transfer catalyst, for example ammonium halide compounds, for instance methyltributylammonium chloride or bromide, tetrabutylammonium chloride or bromide, etc.

Useful solvents are optionally halogenated, aromatic hydrocarbons, for instance toluene, benzene, xylene, or optionally halogenated aliphatic hydrocarbons, DMSO, DMF, acetonitrile or NMP, optionally in combination with water.

Preference is given to using optionally halogenated aromatic hydrocarbons. These are more preferably used in combination with water.

The reaction temperature for this step is from 40 to 110° C., preferably from 60 to 90° C.

To isolate the nitrile, on completion of reaction, the organic phase is removed and the solvent is removed, preferably under reduced pressure.

The remaining distillation bottoms which comprise the desired compound may be used directly for the next step without further purification.

The purity of the nitrile can be increased further if appropriate by distillation or crystallization.

The nitrites of the formula IV are novel and therefore form a further part of the subject matter of the present invention, and also their use for preparing pharmaceuticals and active agrochemical ingredients.

Finally, the nitrile of the formula (IV) is then hydrolyzed to the phenylacetic acid of the formula (Ib) in which R1 is as defined above.

The hydrolysis can be effected in a customary manner either under basic conditions (for example by means of aqueous alkali metal hydroxides) or acidic conditions by means of a customary acid from the group of HCl, H₂SO₄, acetic acid, etc.

Preference is given to performing an acidic hydrolysis. In this hydrolysis, either the distillation bottoms from the second step which comprise the nitrile or further-purified nitrile is used as the starting compound, and is admixed with an acid or an acid mixture in an amount of from 2 to 20, preferably from 5 to 15 equivalents, based on the compound of the formula (IV).

The reaction temperature is from 50 to 120° C.

On completion of reaction, the organic phase is in turn removed and the corresponding phenylacetic acid of the formula (Ib) is obtained by extractive purification in high yields of up to 95% and high purities of up to 98% (HPLC).

The purity can be increased to over 99.55% (HPLC) by recrystallization from an ester, for instance ethyl acetate or isopropyl acetate, etc., or from an ether, for instance diisopropyl ether or MTBE, etc., or a mixture of ester and aliphatic hydrocarbon, for instance heptane, etc.

In variant b), a Friedel-Crafts acylation is first effected with acetyl chloride or acetic anhydride in the presence of a Lewis acid, for example aluminum chloride, iron(III) chloride, tin(IV) chloride or zinc chloride, or a mineral acid as a catalyst, to give the corresponding acetophenone of the formula (V).

Acetyl chloride or acetic anhydride is used in an amount of from 1 to 3 equivalents, preferably of from 1.1 to 2 equivalents, based on the compound of the formula (II).

The amount of catalyst is likewise from 1 to 3 equivalents, preferably from 1.1 to 2 equivalents, based on the compound of the formula (II).

The catalyst used is preferably aluminum chloride. Suitable solvents are optionally halogenated aliphatic hydrocarbons, for instance dichloromethane, chloroform, carbon tetrachloride, etc.

The reaction temperature is from 5 to 40° C., preferably from 15 to 30° C.

After aqueous workup, the corresponding acetophenone of the formula (V) is then obtained in a purity of up to 100% (GC).

In the next step, the acetophenone is converted to the corresponding thioamide of the formula (VI) by a Willgerodt-Kindler reaction with sulfur and an amine of the formula HNR2R3 in which R2 and R3 are each independently a C₁-C₆-alkyl radical or together form a C₂-C₆-alkylene radical which may be interrupted by a heteroatom from the group of O, N or S.

Sulfur and the amine are used in an amount of from 1.5 to 3 equivalents, preferably from 1.8 to 2.5 equivalents, based on the acetophenone.

Suitable amines are, for example, morpholine, dimethylamine, diethylamine, dibutylamine, pyrrolidine, piperidine, etc.

The reaction temperature, depending on the amine used, is from 100 to 180° C., preferably from 120 to 150° C. After the reaction has ended, the reaction mixture is cooled and can be used for the next step without further purification steps.

If appropriate, the corresponding thioamide of the formula (VI) can be purified further by aqueous workup and recystallization.

The thioamides of the formula VI are novel and therefore form a further part of the subject matter of the present invention, and also their use for preparing pharmaceuticals and active agrochemical ingredients.

Finally, analogously to variant a), the thioamide of the formula (VI) is hydrolyzed to the phenylacetic acid of the formula (Ib) in which R1 is as defined above.

The hydrolysis can in turn be effected in a customary manner either under basic conditions (for example by means of aqueous alkali metal hydroxides) or under acetic conditions by means of a customary acid from the group of acetic acid, HCl, H₂SO₄, etc., or combinations thereof.

Preference is given to performing an acetic hydrolysis. The reaction temperature is from 80 to 180° C., preferably from 100 to 150° C.

On completion of reaction, the corresponding phenylacetic acid of the formula (Ib) is obtained by extractive purification in high yields of up to 95% and high purities of up to 98% (HPLC).

The purity can be increased to over 99.5% (HPLC) by recrystallization from an ester, for instance ethyl acetate or isopropyl acetate, etc., or from an ether, for instance diisopropyl ether or MTBE, etc., or a mixture of ester and aliphatic hydrocarbon, for example heptane, etc.

In order to arrive at phenylacetic acids of the formula (I) in which R is C₁-C₄-alkylsulfonyl or C₁-C₄-alkylsulfoxide, the alkylthio radical of the phenylacetic acid of the formula (Ib) obtained by variant a) or b) is converted by oxidation to the corresponding alkylsulfonyl radical in a customary manner, as described, for instance, in WO 04/52869; WO 03/95438, WO 02/46173 or WO 00/58293.

### EXAMPLE 1

### Variant a)

Step 1: Preparation of 3-chloro-4-methylthiobenzyl chloride Starting from 2-chlorothioanisole (Blanc Reaction)

A mixture of 2-chlorothioanisole (200 g, 1.26 mol, 1.00 eq.), paraformaldehyde (126 g, 4.20 mol, 3.33 eq.), ZnCl₂ (75.6 g, 0.55 mol, 0.44 eq.) and hydrochloric acid (630 ml, 37% in H₂O) was stirred at 50° C. for 21 h. The organic phase was removed, washed with water and distilled, which removed unconverted 2-chlorothioanisole (82.4 g, 0.52 mol, 41%). The remaining distillation bottoms consisted for the most part of 3-chloro-4-methylthiobenzyl chloride (123.6 g, purity 83.2% (HPLC), 0.50 mol, yield 39%) and were used directly in the next step. It was also shown that it is possible to increase the purity of this substance further by distillation of 3-chloro-4-methylthiobenzyl chloride (purity: 98.3 a % (GC)).

Step 2: Preparation of 3-chloro-4-methylthio-phenylacetonitrile Starting from 3-chloro-4-methylthiobenzyl chloride (Kolbe Nitrile Synthesis)

A mixture of 3-chloro-4-methylthiobenzyl chloride (100 g, 0.483 mol, 1.00 eq.), NaCN (24.9 g, 0.507 mol, 1.05 eq.), methyltributylammonium chloride (3.80 g, 0.012 mol, 75% in H₂O), H₂O (83 ml) and toluene (150 ml) was stirred at 80° C. for 4.5 h. The organic phase was removed and the solvent was removed under reduced pressure. A dark red, slowly crystallizing melt of 3-chloro-4-methylthiophenylacetonitrile was obtained (104.9 g, purity: 85.84 a % (GC), 0.455 mol, yield: 94%), which was used in the next step without further purification steps.

Kugelrohr distillation of a small portion of the crude product afforded an analytical sample of 3-chloro-4-methylthiophenylacetonitrile in the form of a dark yellow solid. ¹H NMR (300 MHz, CDCl₃): δ=2.47 (2, 3H, SCH₃), 3.69 (s, 2H, 2-H₂), 7.12 (d, 1H, 5′-H), 7.20 (dd, 1H, 6′-H), 7.27 (d, 1H, 2′-H). ¹³C NMR (75 MHz, CDCl₃): δ=15.2 (SCH₃), 22.6 (C-2), 117.3 (C-1), 125.5 (arom.), 126.7 (arom.), 127.3 (arom.), 128.7 (arom.), 132.1 (arom.), 138.2 (arom.). MS: m/z (%)=199, 197 (100) [M]⁺, 162 (49), 150 (48).

Step 3: Preparation of 3-chloro-4-methylthiophenylacetic acid Starting from 3-chloro-4-methylthiophenylacetonitrile

A mixture of 3-chloro-4-methylthiophenylacetonitrile (90.0 g, 0.455 mol, 1.00 eq.) and hydrochloric acid (500 ml, 37% in H₂O) was stirred at 100° C. for 5 h. After removal of the organic phase and extractive purification, 3-chloro-4-methylthiophenylacetic acid (95.0 g, purity: 97.2 a %), (HPLC 0.426 mol, yield: 94%) was obtained in the form of a light brown solid. The purity was increased further by recrystallization from isopropyl acetate, and 3-chloro-4-methylthiophenylacetic acid was obtained in the form of a beige solid (purity: 99.5 a % (HPLC)).

### EXAMPLE 2

### Variant b)

Step 1: Preparation of 3-chloro-4-methylthio-acetophenone Starting from 2-chlorothioanisole (Friedel-Crafts Acylation)

Acetyl chloride (5.10 g, 65 mmol, 1.30 eq.) was added dropwise to a solution, cooled to 0° C., of 2-chloro-thioanisole (7.93 g, 50.0 mmol, 1.00 eq.) and AlCl₃ (10.7 g, 80.0 mmol, 1.60 eq.) in CH₂Cl₂ (100 ml) within 30 min. Subsequently, the mixture was stirred at 23° C. for 21 h. After aqueous workup, 3-chloro-4-methylthioacetophenone was obtained in the form of a gray solid (6.08 g, purity: 100.0 a % (GC), 30.3 mmol, yield: 61%).

Step 2: Preparation of 3-chloro-4-methylthiophenyl-acetic thiomorpholide Starting from 3-chloro-4-methylthioacetophenone (Willgerodt-Kindler Reaction)

A mixture of 3-chloro-4-methylthioacetophenone (6.08 g, 30.3 mmol, 1.00 eq.), sulfur (1.94 g, 60.6 mmol, 2.00 eq.) and morpholine (5.28 g, 60.6 mmol, 2.00 eq.) was stirred at 135° C. for 6 h. After the end of the reaction time, the mixture was cooled and used in the next step without further purification steps.

Aqueous workup of a small portion of the reaction mixture and recrystallization of the crude product from EtOH afforded an analytical sample of 3-chloro-4-methylthiophenylacetic thiomorpholide in the form of a yellow solid. ¹H NMR (300 MHz, CDCl₃): δ=2.40 (s, 3H, SCH₃), 3.46-3.49 (m, 2H, 3″-H₂), 5.59-3.64 (m, 2H, 5″-H₂), 3.71-3.77 (m, 2H, 2″-H₂), 4.27 (s, 2H, 2-H₂), 4.30-4.35 (m, 2H, 6″-H₂), 7.12 (d, 1H, 5′-H), 7.24 (dd, 1H, 6′-H), 7.31 (d, 1H, 2′-H). ¹³C NMR (75 MHz, CDCl₃): δ=15.1 (SCH₃), 49.3, 50.1, 50.8 (C-2, C-2″, C-6″), 66.4, 66.5 (C-3″, C-5″), 125.9 (arom.), 126.7 (arom.), 128.7 (arom.), 132.1 (arom.), 133.4 (arom.), 136.6 (arom.), 199.1 (C-1). MS: m/z (%)=303, 301 (83) [M]⁺, 214 (51), 171 (36), 130 (100), 86 (53).

Step 3: Preparation of 3-chloro-4-methylthiophenyl-acetic acid Starting from 3-chloro-4-methyl-thiophenylacetic thiomorpholide

The reaction mixture from the synthesis of 3-chloro-4-methylthiophenylacetic thiomorpholide was admixed with acetic acid (100 ml) and heated to 120° C. Hydrochloric acid (50 ml, 37% in H₂O) was added and then the mixture was stirred at 120° C. for another 6 h. After extractive purification, 3-chloro-4-methylthiophenylacetic acid was obtained in the form of a light brown solid. The purity was increased further by recystallization from isopropyl acetate, and 3-chloro-4-methylthio-phenylacetic acid was obtained in the form of a beige solid.

1. A process for preparing 3,4-disubstituted phenylacetic acids of the formula (I) 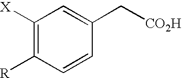
in which X is fluorine, chlorine, bromine or iodine and R is C₁-C₄-alkylthio, C₁-C₄-alkylsulfonyl or C₁-C₄-alkylsulfoxide, wherein a 2-halo-C₁-C₄-alkylthiobenzene of the formula (II) 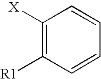
in which X is as defined above and R1 is C₁-C₄-alkylthio
a) is converted by means of the Blanc reaction with formaldehyde and HCl in the presence of a catalyst to the corresponding 3-halo-4-C₁-C₄-alkylthiobenzyl chloride of the formula (III) 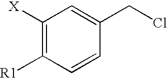
in which X and R1 are each as defined above, which is converted by a Kolbe nitrile synthesis with an alkali metal cyanide to the corresponding phenylacetonitrile of the formula (IV) 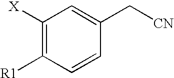
in which X and R1 are each as defined above, which is followed by hydrolysis to the phenylacetic acid of the formula (Ib) 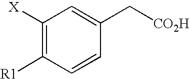
in which X and R1 are each as defined above, or
b) is converted by means of Friedel-Crafts acylation with acetyl chloride or acetic anhydride in the presence of a catalytic acid or a mineral acid as a catalyst to the corresponding acetophenone of the formula (V) 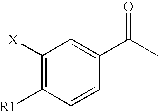
in which X and R1 are each as defined above, which is converted by a Willgerodt-Kindler reaction with sulfur and an amine of the formula HNR2R3 in which R2 and R3 are each independently a C₁-C₆-alkyl radical or together form a C₂-C₆-alkylene radical which may be interrupted by a heteroatom from the group of O, N or S to the corresponding thioamide of the formula (VI) 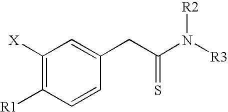
in which X, R1, R2 and R3 are each as defined above, which is then followed by hydrolysis to the phenylacetic acid of the formula (Ib) 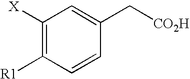
in which X and R1 are each as defined above,
and, if appropriate, after a) or b), the R1 radical of the phenylacetic acid of the formula (Ib) is converted by oxidation to a C₁-C₄-alkylsulfonyl or C₁-C₄-alkylsulfoxide radical.

2. The process as claimed in claim 1, wherein the catalyst used for the Blanc reaction is zinc chloride, aluminum chloride, PCl₃, POCl₃, sulfuric acid or phosphoric acid.

3. The process as claimed in claim 1, wherein the Kolbe nitrile synthesis is performed in the presence of a phase transfer catalyst.

4. The process as claimed in claim 1, wherein the Kolbe nitrile synthesis is performed in an optionally halogenated, aromatic or aliphatic hydrocarbon in combination with water as a solvent.

5. The process as claimed in claim 1, wherein the Friedel-Crafts acylation is performed in an optionally halogenated, aliphatic solvent.

6. The process as claimed in claim 1, wherein morpholine is used as the amine in the Willgerodt-Kindler reaction.

7. The process as claimed in claim 1, wherein the hydrolysis is effected by acidic hydrolysis both in variant a) and b).

8. The process as claimed in claim 1, wherein the purity of the phenylacetic acid of the formula (Ib) obtained by variant a) or b) is increased to over 99.5% by recrystallization from an ester, an ester/aliphatic hydrocarbon mixture or from an ether.

9. A phenylacetonitrile of the formula (IV) 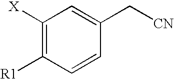
in which X is fluorine, chlorine, bromine or iodine and R1 is C₁-C₄-alkylthio.

10. The use of the compound as claimed in claim 9 for preparing pharmaceuticals and active agrochemical ingredients.

11. A thioamide of the formula (VI) 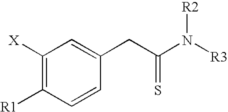
in which X is fluorine, chlorine, bromine or iodine and R1 is C₁-C₄-alkylthio, and R2 and R3 are each independently a C₁-C₆-alkyl radical or together form a C₂-C₆-alkylene radical.

12. The use of the compound as claimed in claim 11 for preparing pharmaceuticals and active agrochemical ingredients.
